(19) United States
(12) Patent Application Publication
(10) Pub. No.: US 2003/0230313 A1
(43) Pub. Date: Dec. 18, 2003

(54) Imaging of the vasa vasorum to navigate through an occlusion

(76) Inventors: Ehsan Alipour, Greenbrae, CA (US); Eric Allison, Rosemount, MN (US); Jonathan A. Mathy, Sugarland, TX (US); Amita Shukla, Ellicott City, MD (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 10/389,294

(22) Filed: Mar. 13, 2003

Related U.S. Application Data
(60) Provisional application No. 60/364,284, filed on Mar. 13, 2002.

(51) Int. Cl. A61B 19/00

(52) U.S. Cl. 128/898; 600/424; 600/470; 604/96.01; 604/101.01

(57) ABSTRACT

An aid in the percutaneous navigation through chronic total occlusions by imaging of the vasa vasorum is provided. A cavity is established in a vessel proximal to the occlusion of interest. Once the cavity is established, fluid is introduced into the cavity. The fluid is introduced under a pressure that is sufficient enough to drive the fluid into the vasa vasorum that is accessible from the cavity. An image is taken with an imaging means of the fluid filled cavity and vasa vasorum. This image is used as a visual aid for a physician to visualize the path/curvature of the vessel. With this visualization, the physician would be able to navigate a medical instrument through an occlusion. In addition, this visualization would aid the physician to open the occlusion and therewith promote blood flow in vessel, for instance, by expanding open, stenting open, or cutting away part of the occlusion.

### CROSS-REFERENCE TO RELATED APPLICATIONS

This application is cross-referenced to and claims priority from U.S. Provisional application No. 60/364,284 filed Mar. 13, 2002, which is hereby incorporated by reference.

### FIELD OF THE INVENTION

The present invention relates generally to medical devices and methods. More particularly, the present invention relates to a device and method to aid in the percutaneous navigation through chronic total occlusions by imaging of the vasa vasorum.

### BACKGROUND

In coronary artery disease, stenosis of arteries can become tighter over time. Eventually, it is possible for a lesion to completely block an artery. When an artery becomes totally blocked over time, the blockage is called a Chronic Total Occlusion (CTO). Opening of a CTO can restore myocardial function to a portion of the heart, and relieve associated angina.

CTOs are generally tough and fibrous, in contrast to the soft blockages caused by acute vulnerable plaque rupture and clotting. Percutaneous devices capable of penetrating, debulking, or dissecting a pathway through the tough matter of CTOs are currently available. However, a principal mode of failure for these devices arises secondary to an inability to safely navigate the device through the occlusion while remaining inside the vessel lumen.

Patients whose occlusions cannot be opened percutaneously must be referred for open heart surgery, with its heightened morbidity and mortality. Accordingly, there is a need for new devices and methods that permit the reliable and safe navigation of percutaneous devices through chronic total occlusions.

### SUMMARY OF THE INVENTION

The present invention provides an aid in the percutaneous navigation through occlusions. In particular, the present invention provides an aid in the percutaneous navigation through CTOs, by imaging of the vasa vasorum. In one aspect of the invention a cavity is established in a vessel proximal to the occlusion of interest. Once the cavity is established, fluid is introduced into the cavity. The fluid is introduced under a pressure that is sufficient to drive the fluid into the vasa vasorum that are accessible from the cavity. The fluid is preferably a contrast agent that is visible to an imaging means. Examples of imaging means are for instance a fluoroscopic image means using a fluoroscopic contrast agent, a Magnetic Resonance Image (MRI) using an MRI contrast agent or an ultrasonic imaging means and using an ultrasound contrast agent. An image is taken with imaging means of at least the fluid filled cavity and vasa vasorum. This image is then used as a visual aid for a physician or interventionist to visualize the path and/or curvature of the occluded vessel. With this visualization, the physician would be able to navigate a medical instrument through the occlusion. In addition, this visualization would aid the physician to open the occlusion and therewith promote blood flow in the vessel, for instance, by expanding open, stenting open, or cutting away part of occlusion. Examples of medical instruments for such interventions that could be used are known in the art and include stents, (laser) guidewires, dissection devices, debulking devices, penetration devices, or the like.

In one embodiment of the invention the means to establish a cavity includes a balloon, which is preferably conformational and capable of creating a circumferential seal with the inner wall of the vessel once the balloon is inflated. Other examples of a means to establish a cavity include means to deploy a diaphragm, a collapsible wedge, or a barrier to retrograde the fluid flow.

In another aspect of the invention a device is provided that includes a first lumen that extends through the means to establish a cavity. In a preferred embodiment, the first lumen extends through the balloon. The first lumen is used to introduce fluid using a fluid delivery means. The fluid delivery means is located outside the body of the patient. The first lumen could also be used to introduce a medical instrument into the cavity for purposes of occlusion intervention. In another embodiment, the device includes a second lumen, which provides an additional channel that could be designated for the delivery of additional medical instruments, as a specifically dedicated channel for medical instruments, or in an additional embodiment as a channel for an extraction means. Extraction means could be used to extract a substance from the cavity such as fluid (contrast agent), blood or occlusion material.

In another embodiment, feedback means could be included to provide feedback of the pressure in the balloon. In yet another embodiment, feedback means could be included to provide feedback of the fluid pressure in the cavity. For instance, a pressure sensor could be used to regulate the pressure in the balloon and cavity, respectively. In still another embodiment each lumen could include a valve, which could also aid in the regulation of fluid flow through the lumen and the fluid pressure in the cavity.

In an alternative embodiment, a device is provided to establish both proximal and distal boundaries of a cavity in a vessel in a patient. This device includes two balloons. A first balloon contains a first lumen which extends through the first balloon, and a second balloon contains a second lumen which extends through the second balloon. A hollow element, such as a tube, is extended from outside the body of the patient and extends through the first lumen as well as through the second lumen. The part of the hollow element situated in between the first balloon and the second balloon has one or more openings. This device includes an inflation means that could inflate the first and second balloon simultaneously or separately. The balloons are also conformational and capable of creating a circumferential seal with the vessel. The device also includes a fluid injection means that is connected to the hollow element to introduce fluid through one or more openings into the cavity.

### BRIEF DESCRIPTION OF THE FIGURES

The objectives and advantages of the present invention will be understood by reading the following detailed description in conjunction with the drawings, in which:

FIG. 1 shows a vessel with a chronic total occlusion;

FIG. 2 shows a vessel with a chronic total occlusion and a device according to the present invention;

FIG. 3 shows a cavity and vasa vasorum filled with a fluid according to the present invention;

FIG. 4 shows an example of a navigation of a medical instrument across a chronic total occlusion using the image of the vasa vasorum as shown in FIG. 3 according to the present invention;

FIG. 5 shows an example of a device with two lumens according to the present invention; and

FIG. 6 shows an alternative embodiment of a device according to the present invention.

### DETAILED DESCRIPTION OF THE INVENTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

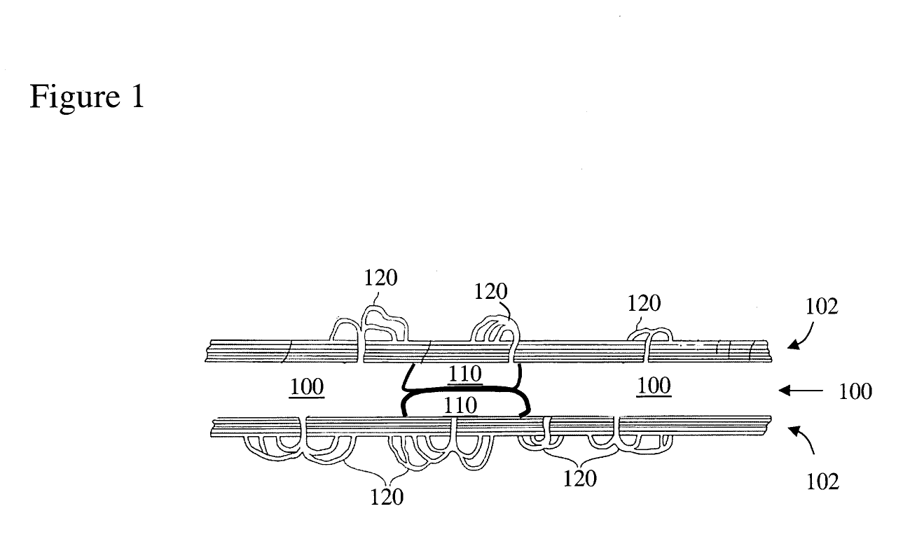

FIG. 1 shows a vessel 100 with a vessel wall 102, a vessel lumen 105 and an occlusion 110, such a chronic total occlusion. The occluded vessel is shown straight in this schematic, but may be bent or convoluted in vivo. Vessel 100 also shows its vasa vasorum 120 at the outside part and through vessel 100, which provides a signature of the path of vessel 100. The key idea of the present invention is to utilize the network of small vessels of vasa vasorum 120 that is situated around occlusion 110 for imaging. This image is then used as a visual aid for a physician or interventionist to visualize the path and/or curvature of vessel 100. With this visualization, the physician would be able to navigate a medical instrument through occlusion 110. In addition, this visualization would aid the physician to open occlusion 110 and therewith promote blood flow in vessel 100, for instance, by expanding open, stenting open, or cutting away part of occlusion 110. Examples of medical instruments for such interventions that could be used are known in the art and include stents, (laser) guidewires, dissection devices, debulking devices, penetration devices, or the like.

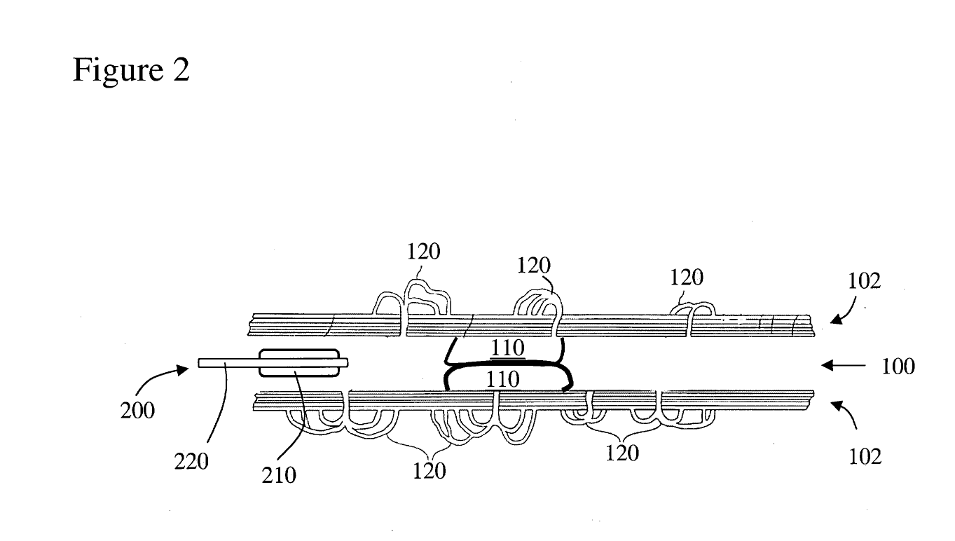

In one aspect of the invention a catheter-based device 200 is entered in vessel 100 as shown in FIG. 2. Device 200 includes a means to establish a cavity 210, such as an inflatable balloon. The balloon could be inflated using an air pump 230 that is positioned outside the body of the patient as is common in the art. The balloon is preferably made of pliable material, conformational (silastic), and capable of creating a circumferential seal with the inner wall of vessel 100 once the balloon is inflated. Device 200 also includes a lumen 220 that is used to introduce a fluid into vessel 100. Lumen 220 is channeled within a flexible intravascular tubing as is common in the art. One end of the tubing and its lumen will remain positioned outside the body of the patient as it is also common in the art. Lumen 220 extends through the balloon or in general terms the means to establish the cavity. The size of lumen 220 is preferably large enough to allow fluid injection and delivery of a medical instrument necessary for interventional procedures of occlusions.

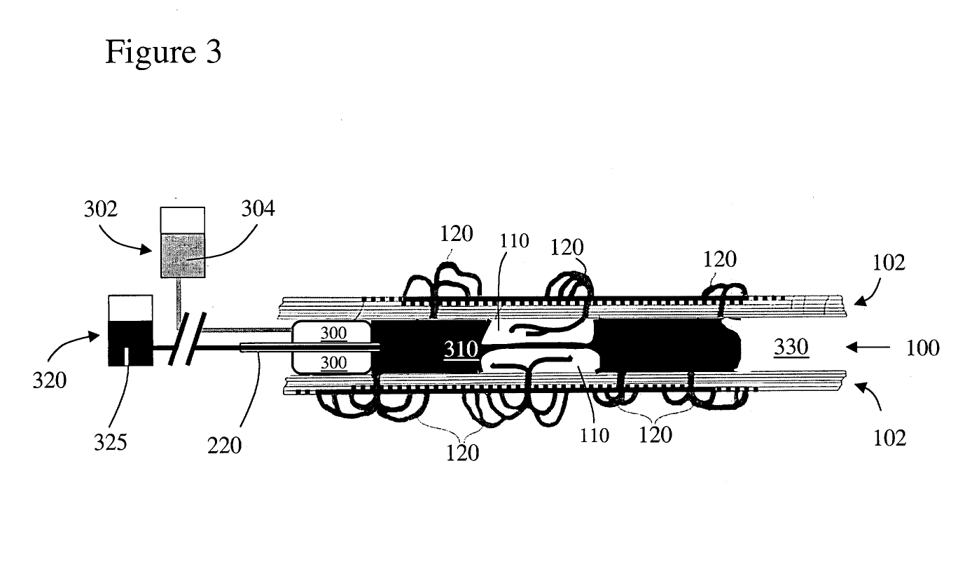

In a preferred aspect of the invention, device 200 is positioned in vessel 100 proximal to occlusion 110 in such a way that a cavity is created in between device 200 and occlusion 110. FIG. 2 shows the balloon in a deflated position, which is preferred to position device 200 in vessel 100 proximal to occlusion 110 (proximal is defined by the direction of blood flow which is also referred to as anterograde movement). FIG. 3 shows the balloon in an inflated position 300 whereby it creates a seal with the inner wall of vessel 100 and a cavity 310 proximal to occlusion 100. The inflation of the balloon could be controlled by a physician or in an automatic fashion by means that are common in the art. FIG. 3 shows an example of a pump 302 to inflate the balloon with for instance saline or air (shown as 304). In one embodiment of the invention, a feedback means (such as a pressure sensor positioned inside the balloon) could be provided to inform the physician or automatic inflation means about the pressure that is building up in the balloon as well as enabling regulation over the pressure in the balloon.

Lumen 220 is connected to a means to introduce fluid 320 in cavity 310 as shown in FIG. 3. Such a means 320 is preferably positioned outside the body of the patient. A physician controls the amount of fluid 325 that needs to be introduced in cavity 310, however this could also be controlled in an automatic fashion. In one embodiment of the invention, a feedback means (such as a pressure sensor positioned at the cavity site of the catheter) could be provided to inform the physician or automatic fluid injection means about the pressure that is building up in cavity 310 as well as enabling regulation over the pressure in cavity 310. The key idea of introducing fluid in cavity 310 is to build up enough pressure so that at least cavity 310 and vasa vasorum 120 receive fluid as shown in FIG. 3. The pressure should be sufficient enough to drive or diffuse an anterograde movement of the fluid through vasa vasorum 120. In some cases, the fluid may re-enter the vessel lumen distal 330 to the occlusion or perfuse the tissue of the occlusion itself, as well as other adjacent structures which may communicate with the vasa vasorum.

The fluid is preferably a contrast fluid that could be used in conjunction with an imaging device (not shown). In one aspect of the invention a fluoroscopic imaging means is used and the fluid is a fluoroscopic contrast agent. However, the present invention is not restricted to the use of fluoroscopic imaging means and a fluoroscopic contrast agent as one could also use Magnetic Resonance Imaging (MRI) and MRI contrast agents, ultrasound and ultrasound contrast agents, or other techniques that are common in the medical art. Imaging means should be capable of making an image of at least the vasa vasorum and cavity both filled with the fluid irrespective of the skin and other subcutaneous tissue and/or organs that are located in between the imaging device and area of the vasa vasorum and cavity. The image could also include structures that are in communication with the vasa vasorum such as the capillary structures within the occluding lesion, adjacent areas of normal and pathological vessel wall, as well as vessel lumen distal to the site of occlusion.

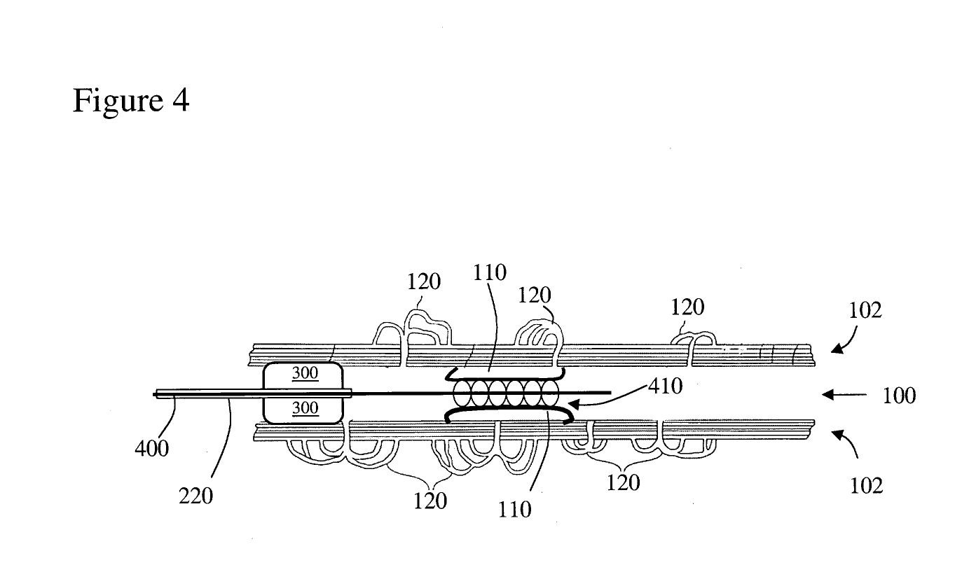

FIG. 4 shows an example of a navigation of a medical instrument across an occlusion using an image based on the vasa vasorum that has been filled with, for example, a fluoroscopic, MRI contrast agent or ultrasound contrast agent. Medical instruments that are suitable are typically the ones that are used for occlusion intervention and small enough to fit through the lumen of the balloon. The example of FIG. 4 shows the navigation of a guidewire 400 across occlusion 110 by a physician guided by an image of the vasa vasorum as shown in FIG. 4. This could either be accomplished alone or in conjunction with another device such as a dissection device, laser guidewire, rotablator, or the like. With guidewire 400 in place, the physician can place a stent 410 to open occlusion 110, thereby restoring blood flow to vessel 100.

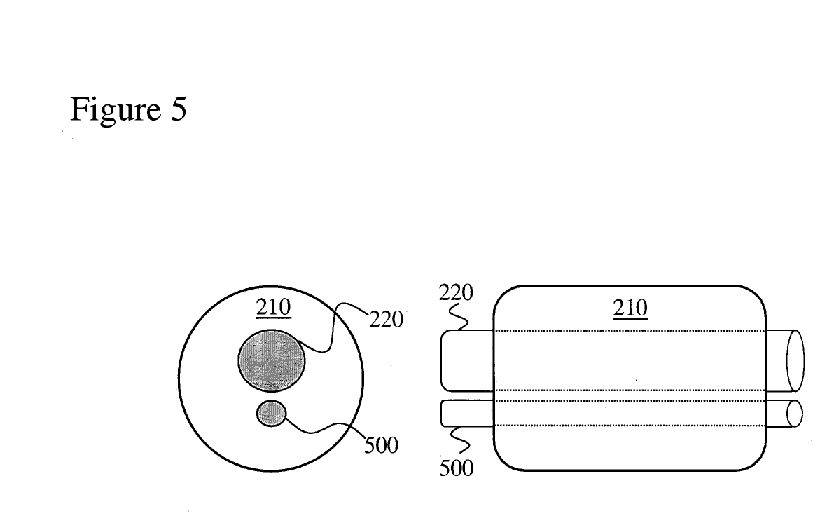

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For instance, the medical instrument could be entered through the same lumen as used for injecting the fluid. However, in another embodiment of the invention a second lumen 500 could be used for introducing or delivering a medical instrument that is also extended through the balloon 510 (i.e. means to establish a cavity) as shown in FIG. 5. In yet another embodiment of the invention, the first or second lumen could be used to extract fluid, blood or material from the occlusion using an extraction means (not shown). An example of an extraction means is, for instance, a suction device positioned outside the body of the patient, as it is common in the art. In yet another aspect of the invention, each lumen could include a valve (one-way or two-way valve, not shown) to prevent pressure drop through leakage of the injected fluid as well as to provide control over flow direction. In the exemplary embodiments described supra, a balloon was used as a means to create a cavity. However, the present invention is not limited to a balloon and could also include a diaphragm, collapsible wedge, deployable barriers, or the like.

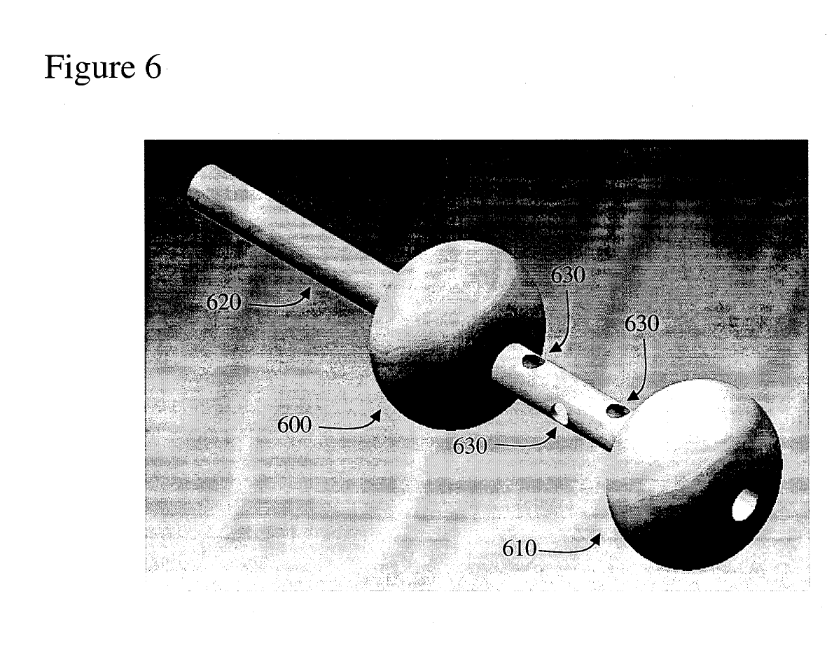

An alternative embodiment includes a catheter-based device with a first balloon 600 and a second balloon 610 as shown in FIG. 6. Each balloon can be inflated at the same time with a shared inflation means or inflated independently each with its own inflation means. The inflation of balloons 600 and 610 occurs in a similar fashion as described supra. The second balloon 610 is positioned at a proximal position along, for instance, a percutaneous tubing 620. The present invention is not limited to percutaneous tubing since various kinds of cylinders or hollow elements could be used that are able to deliver a fluid. In this example the second balloon 610 is used to create a cavity in between both balloons in a similar fashion as described supra. This would be useful in circumstances where a total occlusion is not present or not convenient for utilization as one boundary of the cavity. Fluid can be introduced in the established cavity in a similar fashion as discussed supra whereby the fluid is delivered through percutaneous tubing 620 and through holes 630 into the established cavity. All such variations are considered to be within the scope and spirit of the present invention as defined by the following claims and their legal equivalents.

What is claimed is:

1. A method to navigate across an occlusion, comprising the steps of:
(a) creating a cavity in a vessel, wherein said vessel comprises said occlusion and wherein said cavity is created proximal to said occlusion;
(b) introducing a fluid into said cavity, wherein said fluid is introduced under pressure sufficient enough to drive said fluid into the vasa vasorum accessible from said cavity;
(c) making an image of said introduced fluid in said cavity and said vasa vasorum; and
(d) navigating through said occlusion with a medical instrument using said image.

2. The method as set forth in claim 1, wherein said step of creating a cavity comprises the step of inflating a balloon, wherein said balloon is conformational and capable of creating a circumferential seal with said vessel, deploying a diaphragm, deploying a collapsible wedge, or deploying a barrier to retrograde said fluid flow.

3. The method as set forth in claim 2, wherein said fluid is introduced through a first lumen, wherein said first lumen extends across said balloon.

4. The method as set forth in claim 2, wherein said medical instrument is navigated through a first lumen, wherein said first lumen extends across said balloon.

5. The method as set forth in claim 1, wherein said step of creating a cavity comprises the step of inflating a balloon and wherein said step of navigating through said occlusion with said medical instrument comprises the step of delivering said medical instrument through a second lumen, wherein said second lumen extends across said balloon.

6. The method as set forth in claim 5, wherein said medical instrument comprises a stent, a guidewire, a debulking device, dissection device, or penetrating device.

7. The method as set forth in claim 1, further comprising the step of providing a feedback means to provide feedback of said pressure in said cavity and to regulate said pressure in said cavity.

8. The method as set forth in claim 1, further comprising the step of providing a feedback means to provide feedback of said pressure in said means to create a cavity and to regulate said pressure in said means to create a cavity.

9. The method as set forth in claim 1, wherein said image is a fluoroscopic image and said fluid comprises a fluoroscopic contrast agent, an MRI and said fluid comprises an MRI contrast agent, or an ultrasonic image and said fluid comprising an ultrasound contrast agent.

10. The method as set forth in claim 1, further comprising the step of providing an extraction means to extract substance from said cavity, wherein said substance is fluid, blood or occlusion material.

11. A device for navigating a medical instrument across an occlusion wherein said occlusion is located in a vessel, comprising:
(a) a means to establish a cavity in said vessel proximal to said occlusion;
(b) a first lumen extended through said means to establish a cavity; and
(c) a fluid injection means to introduce fluid through said first lumen, wherein said fluid is introduced under pressure sufficient enough to drive said fluid into the vasa vasorum accessible from said cavity.

12. The device as set forth in claim 11, wherein said first lumen allows delivery of said medical instrument.

13. The device as set forth in claim 11, further comprising a second lumen extended through said means to establish a cavity to allow passage of said medical instrument.

14. The device as set forth in claim 13, wherein said second lumen comprises a valve to regulate said pressure in said cavity.

15. The device as set forth in claim 11, wherein said first lumen comprises a valve to regulate said pressure in said cavity.

16. The device as set forth in claim 11, further comprising an extraction means to extract substance from said cavity, wherein said substance is fluid, blood or occlusion material.

17. The device as set forth in claim 11, further comprising a feedback means to provide feedback of said pressure in said cavity and to regulate said pressure in said cavity.

18. The device as set forth in claim 11, further comprising a feedback means to provide feedback of said pressure in said means to establish a cavity and to regulate said pressure in said means to create a cavity.

19. The device as set forth in claim 11, wherein said means to establish a cavity comprises an inflatable balloon wherein said balloon is conformational and capable of creating a circumferential seal with said vessel, deploying a diaphragm, deploying a collapsible wedge, or deploying a barrier to retrograde said fluid flow.

20. A system for navigating a medical instrument across an occlusion wherein said occlusion is located in a vessel, comprising:
(a) a means to establish a cavity in said vessel proximal to said occlusion, wherein said means to define a cavity comprises a first lumen extended through said means to define a cavity;
(b) a means to introduce fluid through said first lumen, wherein said fluid is introduced under pressure sufficient enough to drive said fluid into the vasa vasorum accessible from said cavity;
(c) a means to image said injected fluid in said cavity and said vasa vasorum; and
(d) a means to navigate across said occlusion with said medical instrument using said image as guidance.

21. The system as set forth in claim 20, wherein said first lumen allows for delivery of said medical instrument.

22. The system as set forth in claim 20, wherein said means to establish a cavity further comprises a second lumen extended through said means to establish a cavity, wherein said second lumen allows for delivery of said medical instrument.

23. The system as set forth in claim 20, further comprising an extraction means to extract substance from said cavity, wherein said substance is fluid, blood or occlusion material.

24. The system as set forth in claim 20, wherein said means to image comprises a fluoroscopic contrast agent, an MRI and said fluid comprises an MRI contrast agent, or an ultrasonic imaging and said fluid comprising an ultrasound contrast agent.

25. A device for establishing a cavity in a vessel in a patient, comprising:
(a) a first balloon with a first lumen extended through said first balloon;
(b) a second balloon with a second lumen extended through said second balloon; and
(c) a hollow element extended from outside the body of said patient through said first lumen and through said second lumen, wherein the part of said hollow element in between said first balloon and said second balloon comprises one or more openings.

26. The device as set forth in claim 25, further comprising an inflation means, wherein said inflation means inflates said first balloon or said second balloon, or inflates said first balloon and said second balloon.

27. The device as set forth in claim 25, wherein said inflated first and second balloon are conformational and are capable of creating a circumferential seal with said vessel.

28. The device as set forth in claim 25, further comprising a fluid injection means connected to said hollow element to introduce fluid through said one or more openings in said cavity.
